(19) United States
(12) Patent Application Publication
(10) Pub. No.: US 2008/0003226 A1
(43) Pub. Date: Jan. 3, 2008

(54) Method to Reduce Hepatoxicity of Fas-Mediated Apoptosis-Inducing Agents

(75) Inventors: Marc Dupuis, Blonay (CH); Stephane Demotz, Epalinges (CH); Rene Goedkoop, Bogis Bossey (CH)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(73) Assignee: APOXIS SA, EPALINGES (CH)

(21) Appl. No.: 11/570,033

(22) PCT Filed: May 27, 2005
(86) PCT No.: PCT/EP05/52436

§ 371 Date: Dec. 4, 2006

Related U.S. Application Data
(60) Provisional application No. 60/577,399, filed on Jun. 4, 2004.

(51) Int. Cl. A61K 39/395; A61P 43/00

(52) U.S. Cl. 424/145.1

(57) ABSTRACT

The invention concerns a method to prevent or reduce adverse effects on liver of a patient treated with a Fas-mediated apoptosis-inducing agent, the method comprising the administration of a product preventing TNF receptors-mediated apoptosis of the liver cells.

The present invention relates to a new method to reduce hepatotoxicity of Fas-mediated apoptosis-inducing agents.

It is known that liver is particularly susceptible to Fas-mediated cytotoxicity. Due to their efficacy, some Fas-mediated apoptosis inducers may present high risk of liver toxicity (DeVries & al. Drugs Today (Barc). 2003; 39 Suppl C:95-109). Administration of agonistic anti-Fas antibodies to mice may lead to acute liver failure and death within a few hours (Ni & al., 1994, Exp. Cell. Res. 215, 332-337; Ogaswara & al., 1993, Nature 364, 806-809).

It has been also discovered that toxicity of multimerized forms of soluble Fas ligands depends on the route of administration. In particular, intravenous injection of 50 μg/kgMega-FasL (hexamer of extracellular domain of FasL fused to an ACRP30 fragment) to mouse, results in death within 2 to 6 hours through liver failure. By contrast, intraperitoneal injection of that dose of Mega-FasL will not cause death (EP 032912473, incorporated herein by reference). Indeed, death is eventually observed following intraperitoneal injection of doses several fold higher (100-200 μg/kg).

The present invention relates to a new method to prevent or reduce adverse effects of the liver in patients treated with a Fas-mediated apoptosis inducing agent. The method comprises the administration of a product preventing TNF receptors-mediated apoptosis of the liver cells. TNF receptors are selected among TNFR1 and TNFR2.

The product preventing TNF receptors-mediated apoptosis preferably prevent release of TNF and/or lymphotoxins, or preferably is an anti-TNF/TNFR interaction product preventing TNF-mediated apoptosis of the liver cells.

Said administration can be made according to the invention sequentially, separately or simultaneously, with the Fas-mediated apoptosis inducing agents.

Sequential treatment means according to the invention either a pre-treatment with the product preventing TNF receptors-mediated apoptosis, prior treatment with the Fas-mediated apoptosis inducing agent or a post treatment with the product preventing TNF receptors-mediated apoptosis, after and during treatment with the Fas-mediated apoptosis inducing agent, and their combinations thereof.

The pre-treatment will prevent TNF-mediated apoptosis prior treatment with the Fas-mediated apoptosis inducing agent.

The post-treatment may be initiated based on the detection of increased liver enzymes concentrations, such as ALAT and ASAT, after treatment with the Fas-mediated apoptosis inducing agent, to reduce toxicity when detected.

Alternatively, pre-treatment may be followed by post treatment.

When used sequentially or separately, Fas-mediated apoptosis inducing agent and product preventing TNF receptors-mediated apoptosis are used in separate pharmaceutical compositions, each of them being appropriate for the chosen administration route. Both compositions may, however, be combined in the same package or treatment-kit.

When used simultaneously, Fas-mediated apoptosis inducing agent and product preventing TNF receptors-mediated apoptosis may be combined in the same pharmaceutical compositions appropriate for the chosen administration route.

Pharmaceutical compositions comprising a Fas-mediated apoptosis inducing agent and a product preventing TNF receptors-mediated apoptosis as well as treatment kits comprising in separate pharmaceutical compositions a Fas-mediated apoptosis inducing agent and a product preventing TNF receptors-mediated apoptosis are also part of the present invention.

Suitable pharmaceutical compositions and carriers, adjuvant, preservatives, etc., used to prepare pharmaceutical compositions, are well-known to those in the art (see Gennaro (ed.), Remington's Pharmaceutical Sciences, 19th Edition (Mack Publishing Company 1995)).

According to the present invention, Fas-mediated apoptosis inducing agents comprise agonistic Fas antibodies and soluble FasL molecules or multimers there off.

Diseases or pathologies treated with the method according to the present invention include all pathologies comprising cell proliferation. It includes more particularly pathologies where cell death has to be induced for its control and/or treatment, such as primary and secondary cancer and its metastases, as well as haematological malignancies, more particularly mesotheliomas, ovarian cancers, pancreas cancers, prostate cancers, breast cancers, non small cell lung cancers, melanomas, colon carcinoma, glioblastomas, myelomas and leukemias.

Sensitivity of different tumor cell lines to apoptosis by hexamers of FasL is givent in the table below.

| | Sensitivity |
|---|---|
| Mesotheliomas | |
| H28 | ++ |
| H2052 | − |
| H2373 | ++ |
| H2452 | ++ |
| SPC111 | ++ |
| SPC212 | − |
| ZL5 | + |
| ZL34 | ++ |
| ZL55 | +/− |
| H-Meso-1 | +/− |
| Met5A | + |
| Ovarian cancers | |
| OVCAR-3 | + |
| SKOV-3 | + |
| Pancreas cancer | |
| CAPAN-2 | ++ |
| Prostate cancers | |
| BxPC3 | ++ |
| PC3 | ++ |
| Breast cancer | |
| MCF-7 | ++ |
| Non small cell lung cancers | |
| H226 | ++ |
| A-549 | ++ |
| Melanomas | |
| C-32 | ++ |
| HT-144 | ++ |
| Colon carcinoma | |
| HT29 | ++ |
| Glioblastomas | |
| U87 | ++ |
| U251 | ++ |
| LN229 | + |
| Myelomas | |
| RPMI8226 | ++ |
| U266 | + |
| Leukemias | |
| HL60 | + |
| K562 | − |

Agonist Fas antibodies are known in the art, including antibodies disclosed in Tinel & al. (Hepatology. 2004 March; 39(3):655-66), Rubio Pomar & al. (Biol Reprod. 2004 May 5), Pechelet & al. (Biochem Pharmacol. 2004 Feb 1; 67(3):523-37), Clarke & al. (Haematologica. 2004 January; 89(1):11-20), Imai & al. (Oncogene. 2003 Dec. 18; 22(58):9231-42), Legembre & al. (J Immunol. 2003 Dec. 1; 171(11):5659-62), Chun & al. (Toxicol Lett. 2003 Dec. 15; 146(1):75-81), which content is incorporated herein by reference.

Soluble FasL molecules comprise monomers, oligomers and multimers of FasL molecules, more particularly of the soluble, extracellular domain of the ligand. In a preferred embodiment, the soluble FasL molecule is selected among multimerized FasL molecules.

The multimerized FasL molecules according to the invention comprise at least four, globular soluble extracellular fractions of the Fas ligand, preferably at least five, more preferably at least six, even more preferably six globular soluble extracellular fractions of the Fas ligand bound to a multimerization moiety.

Multimerized FasL molecules may eventually aggregate to form higher degrees of multimerization, including dodecamer (2 hexamers) or octodecamers (3 hexamers).

In a preferred embodiment of the invention, the multimerized form of Fas ligand of is a hexamer comprising six monomers, assembled together, each of the monomers comprising a polypeptide of formula (1):
H-L (1)
wherein:
L represents a C-terminal Fas ligand moiety, comprising the soluble extracellular fraction of a Fas ligand, and
H represents an N-terminal hexamerization moiety.

According to the present invention, the ligand moiety L includes the “full length” of the soluble extracellular fraction of Fas ligand and biologically functional fragments of the same fraction. “Biologically functional fragments” are fragments of a soluble extracellular fraction of a ligand of the TNF family conserving their ability to bind to the same receptor(s), with substantially the same affinity.

L is preferably comprises the full length extracellular soluble fraction of the above ligand.

According to an embodiment of the invention, L comprises the extracellular domain of human FAS ligand (hFasL), comprising amino acids Glu 139 to leu 281 of hFasL.

Hexamers according to the invention are either “true” hexamers, dimers of trimers or trimers of dimers. In the first case, H is a hexamerization polypeptide HP. In the latter cases, H comprises two moieties, a first moiety consisting of a dimerization polypeptide (DP) and a second moiety consisting of a trimerization polypeptide (TP).

The polypeptides according to the present invention comprise a polypeptide represented by one the following formulas (Ia), (Ib) and (Ic):
HP-L (Ia)
(“true” hexamers),
DP-TP-L (Ib)
(trimers of dimers), and
TP-DP-L (Ic)
(dimers of trimers)
wherein L, HP, DP and TP are defined above and below.

Examples of HP, TP and DP are well known in the art and comprise isolated peptide fragments of natural hexameric, trimeric or dimeric polypeptides, the said isolated fragments being responsible for the hexamerization, dimerization or trimerization of the said natural hexamers, dimers or trimers.

Such molecules are well known in the art and comprises polypeptides of the collectin family, such as the ACRP30 or ACRP30-like proteins (WO96/39429, WO 99/10492, WO 99/59618, WO 99/59619, WO 99/64629, WO 00/26363, WO 00/48625, WO 00/63376, WO 00/63377, WO 00/73446, WO 00/73448 or WO 01/32868), apM1 (Maeda et al., Biochem. Biophys. Res. Comm. 221: 286-9, 1996), C1q (Sellar et al., Biochem. J. 274: 481-90, 1991), or C1q like proteins (WO 01/02565), which proteins comprise “collagen domains” consisting in collagen repeats Gly-Xaa-Xaa′.

Other oligomerized polypeptides are known in the art, including polypeptides with a “coiled-coil” domains (Kammerer R A, Matrix Biol 1997 March; 15(8-9):555-65; discussion 567-8; Lombardi & al., Biopolymers 1996; 40(5):495-504; http://mdl.ipc.pku.edu.cn/scop/data/scop.1.008.001.html), like the Carilage Matrix Protein (CMP) (Beck & al., 1996, J. Mol. Biol., 256, 909-923), or polypeptides with a dimerization domain, like polypeptides with a leucine zipper or osteoprotegerin (Yamaguchi & al., 1998).

According to a specific embodiment of the invention, HP comprises the hexamerization domains of A, B or C chains of polypeptides of the C1q family.

TP are known in the art and comprise the trimerization domains (C-terminal moiety) of CMP (i.e. GeneBank 115555, amino acids 451-493) or the trimerization domain of ACRP30 and ACRP30-like molecules. According to a preferred embodiment of the present invention, TP comprises a stretch of collagen repeats.

According to the invention, a “stretch of collagen repeats” consists in a series of adjacent collagen repeats of formula (II):
-(Gly-Xaa-Xaa′)ₙ- (II)
wherein:
Xaa and Xaa′ represents independently an amino acid residue, and
n represents an integer from 10 to 40.

Xaa and Xaa′ are preferably selected independently among natural amino acids such as Ala, Arg, Asn, Asp, Cys, Gln, Glu, Gly, His, Ile, Leu, Lys, Met, Phe, Pro, Ser, Thr, Trp, Tyr or Val.

Xaa preferably represents independently an amino acid residue selected among Ala, Arg, Asp, Glu, Gly, His, Ile, Leu, Met, Pro or Thr, more preferably Arg, Asp, Glu, Gly, His or Thr.

Xaa′ preferably represents independently an amino acid residue selected among Ala, Asn, Asp, Glu, Leu, Lys, Phe, Pro, Thr or Val, more preferably Asp, Lys, Pro or Thr.

When Xaa′ represents a Pro residue, the collagen repeat Gly-Xaa-Pro is designated to be a “perfect” collagen repeat, the other collagen repeats being designated as “imperfect”.

According to a preferred embodiment of the invention, the stretch of collagen repeats comprises at least 1 perfect collagen repeat, more preferably at least 5 perfect collagen repeats.

According to a preferred embodiment of the invention, n is an integer from 15 to 35, more preferably from 20 to 30, most preferably 21, 22, 23 or 24.

According to the present invention, the stretch of collagen repeat may comprise up to three “non collagen residues” inserted between two adjacent collagen repeats. These “non collagen residues” consist in 1, 2 or 3 amino acid residues, provided that when the “non collagen residue” consists in 3 amino acids residues, the first amino acid is not Gly.

According to a preferred embodiment of the invention, TP consists in an uninterrupted stretch of 22 collagen repeats. More preferably, TP consists in the stretch of 22 collagen repeats of SEQ ID NO 1, corresponding to amino acids 45 to 110 of mACRP30, as represented in SEQ ID NO 2 of WO 96/39429:

Gly Ile Pro Gly His Pro Gly His Asn Gly Thr Pro Gly Arg Asp Gly Arg Asp Gly Thr Pro Gly Glu Lys Gly Glu Lys Gly Asp Ala Gly Leu Leu Gly Pro Lys Gly Glu Thr Gly Asp Val Gly Met Thr Gly Ala Glu Gly Pro Arg Gly Phe Pro Gly Thr Pro Gly Arg Lys Gly Glu Pro Gly Glu Ala

According to another preferred embodiment of the invention, TP consists in the stretch of 22 collagen repeats corresponding to amino acids 42 to 1107 of hACRP30, as represented in SEQ ID NO 7 of WO 96/39429:

DP are known in the art and comprises dimerization fragments of immunoglobulins (Fc fragments), the C-terminal dimerization domain of osteoprotegerin (Receptor: δN-OPG; amino acids 187-401), or polypeptides sequences comprising at least 6, preferably 8 to 30 amino acids and allowing dimerization. These peptides generally comprise at least a cysteine residue allowing the formation of disulfide bonds. Other polypeptides useful as DP according to the invention are peptides designated as “leucine zippers” comprising a Leucine residue being present every seventh residue.

Examples of such peptides comprising at least a cysteine residue comprise the following peptides:

Val Asp Leu Glu Gly Ser Thr Ser Asn Gly Arg Gln Cys Ala Gly Ile Arg Leu

Glu Asp Asp Val Thr Thr Thr Glu Glu Leu Ala Pro Ala Leu Val Pro Pro Pro Lys Gly Thr Cys Ala Gly Trp Met Ala

Gly His Asp Gln Glu Thr Thr Thr Gln Gly Pro Gly Val Leu Leu Pro Leu Pro Lys Gly Ala Cys Thr Gly Trp Met Ala.

The second sequence above corresponds to amino acids 17 to 44 of mACRP30 as represented in SEQ ID NO 2 of WO 96/39429, and the third sequence above corresponds to amino acids 15 to 41 of SEQ ID NO 7 of WO 96/39429.

Other peptides comprising at least one cysteine residue, can be found in amino acid sequences upstream the stretch of collagen repeats of molecules having a structure analogous to ACRP30 (ACRP30-like) as disclosed in WO 99/10492, WO 99/59618, WO 99/59619, WO 99/64629, WO 00/26363, WO 00/48625, WO 00/63376, WO 00/63377, WO 00/73446, WO 00/73448 or WO 01/32868.

Leucine zippers are well known in the art and can be found in natural proteins and eventually identified using bioinformatics tools available to the one skilled in the art (http://www.bioinf.man.ac.uk/zip/faq.shtml; http://2zip.molgen.mpg.de/; Hirst, J. D., Vieth, M., Skolnick, J. & Brooks, C. L. III, Predicting Leucine Zipper Structures from Sequence, Protein Engineering, 9, 657-662 (1996)).

The constitutive elements L, H, HP, TP and/or DP in the polypeptides of formula I, Ia, Ib or Ic, according to the invention, are assembled by peptides bonds. They may be separated by “linkers” who will not affect the functionality of the polypeptide according to the invention, its ability to form hexamers and to bind with the receptor corresponding to the ligand L. Such linkers are well known in the art of molecular biology.

The polypeptide according to the invention may also comprise peptide sequences on its N-terminus and/or C-terminus, which will not affect the functionality of the polypeptide according to the invention. These peptides may comprise affinity tags, for purification or detection of the polypeptide according to the invention. Such affinity tags are well known in the art and comprise a FLAG peptide (Hopp et al., Biotechnology 6: 1204 (1988)) or a Myc-His tag.

According to a preferred embodiment of the invention, H comprises a dimerization polypeptide (DP) and a trimerization polypeptide (TP), and is most preferably represented by the following formula:
DP-TP-L (Ib)
wherein R, DP and TP are defined above and below.

More preferably, DP and TP represent together amino acids 17 to 110 of mACRP30 as represented in SEQ ID NO 2 of WO 96/39429 or amino acids 15 to 107 of hACRP30 as represented in SEQ ID NO 7 of WO 96/39429.

As a preferred embodiment of the invention the polypeptide comprises the fusion polypeptide m or hACRP30:hFasL (MegafasL), more particularly m or hACRP30:hFasL disclosed in WO 01/49866 which content is incorporated herein by reference.

According to another embodiment of the invention, the hexamerization moiety comprises a Fc portion of IgG comprising amino acids 248 to 473 of gi2765420, as disclosed in the PCT application No. PCT/EP02/09354, which content is incorporated herein by reference.

Products preventing TNF receptors-mediated apoptosis of the liver cells, and more particularly anti-TNF/TNFR interaction products preventing TNF-mediated apoptosis are also known in the art. They include known anti-tumor agents such as anti-TNF antibodies and soluble TNF receptors. They also include small molecular weight molecules interfering with TNF activity, such as thalidomide or suramin. They also include inhibitors of other ligands of the TNFR1 and TNFR2, such as lymphotoxin-alpha.

Anti-TNF antibodies are known in the art. In a preferred embodiment, the anti-TNF antibody is a monoclonal antibody, more preferably a recombinant monoclonal antibody such as infliximab, sold under the trade name Remicade by Centocor.

Soluble receptors comprise preferably the soluble extracellular fraction of the TNF receptor. In a preferred embodiment, the soluble TNF receptor is a multimer, such as a tetradrimer, a pentamer (WO 01/49866) or a hexamer (WO 03/095489). In a preferred embodiment, the soluble receptor is etanercept, sold under the trade name Embrel by Wyeth.

In the method according to the invention, treatment with Fas-mediated apoptosis inducing agents may be combined with other means of treatment comprising other molecules or compositions suitable for the treatment of the same diseases, but also other means of treatment known in the art of treatment of the same diseases such as radiation therapy, chemotherapy, or eventually surgery.

Other molecules or compositions suitable for the treatment of the same diseases are well known in the art, such as any of the molecules or compositions listed under the heading “Cancerologie” in the Dictionaire Vidal (2003 ed.), in the Merk Index or in the Physician Desk Reference.

Use of the Fas-mediated apoptosis inducing agents defined above and more particularly hexamers of the soluble fraction of FasL as defined above as an adjuvant with existing chemotherapies is another embodiment of the present invention. As an example, cell death was greatly increased when MegaFasL was combined with cisplatin, when only marginal cell death was measured with either of these compounds alone. Actually, the antitumor effectiveness of MegaFasL as single agent and in combination with cisplatin was evaluated first on mesothelioma and ovarian cancer cell lines in vitro. Cytotoxicity experiments showed that the pretreatment of cells with sub-toxic doses of chemotherapy up to 3 days before treatment with MegaFasl induced a strong synergistic effect between the treatments.

The present invention also concerns the use of a product preventing TNF/lymphotoxin-mediated apoptosis of the liver cells as defined above for the preparation of a medicament used in the prevention or reduction of adverse effects on liver of a patient treated with a Fas-mediated apoptosis-inducing agent as defined above.

The present invention also concern the use of a product preventing TNF/lymphotoxin-mediated apoptosis of the liver cells as defined above and a Fas-mediated apoptosis-inducing agent as defined above for the preparation of a medicament for the treatment of cancer.

The present invention also concern a product comprising a product preventing TNF receptors-mediated apoptosis of the liver cells as defined above and a Fas-mediated apoptosis-inducing agent as defined above for use simultaneously, separately or sequentially, in the treatment of pathologies where cell death has to be induced for its control and/or treatment as defined above.

The following treatments may be conducted according to the invention.

The patient who suffers from some advanced stage malignancies (mesothelioma or ovary cancer) is first repeatedly injected with Embrel. Alternatively, he/she is administered some standard thalidomide treatment, such as those prescribed for the therapy of inflammatory conditions. Then, the patient is treated with standard chemotheraypy (i.e. cis-platinum or 5-fluorouracil) before starting administration with Mega-FasL (intravenous or intraperitoneal, bolus injection or perfusion).

The following ongoing in vivo experiments are performed.

1) TNFR1 and TNFR2 double knocked-out mice and control wild type mice are injected with increasing doses of Mega-FasL. ALAT and ASAT levels and survival are monitored. It is expected that the TNF receptor-deficient mice will show a lower susceptibility to liver toxicity than normal mice.

2) Normal mice will be administered with recombinant TNFR1-Fc fusion protein (neutralizes TNF-alpha and lymphotoxin-alpha) before injection of increasing doses of Mega-FasL. ALAT and ASAT levels and survival are monitored. It is expected that mice treated with TNFR1-Fc protein will show a lower susceptibility to liver toxicity than untreated mice.

3) Normal mice will be administered with suramin or thalidomide. Subsequently, the animals will be injected with increasing doses of Mega-FasL. ALAT and ASAT levels and survival will be then monitored over time. It is expected that mice treated with thalidomide or suramin will show a lower susceptibility to Mega-FasL-induced liver toxicity than untreated mice (Eichhorst et al. 2004. Suramin inhibits death receptor-induced apoptosis in vitro and fulminant apoptotic liver damage in mice. Nature Medicine 10:602).

1. A method to prevent or reduce adverse effects on liver of a patient treated with a Fas-mediated apoptosis-inducing agent, the method comprising the administration of a product preventing TNF receptors-mediated apoptosis of the liver cells, said administration being made sequentially, separately or simultaneously, with the Fas-mediated apoptosis inducing agents.

2. (canceled)

3. The method of claim 1, wherein sequential treatment is either a pre-treatment with the product preventing TNF receptors-mediated apoptosis of the liver cells prior treatment with the Fas-mediated apoptosis inducing agent or a post treatment with the product preventing TNF receptors-mediated apoptosis of the liver cells, after and during treatment with the Fas-mediated apoptosis inducing agent, and their combinations thereof.

4. The method as claimed in claim 3, wherein pre-treatment shall prevent TNF/lymphotoxin-mediated apoptosis prior treatment with the Fas-mediated apoptosis-inducing agent.

5. The method as claimed in claim 3, wherein post-treatment is initiated based on the detection of increased liver enzymes concentrations, such as ALAT and ASAT, after treatment with the Fas-mediated apoptosis inducing agent, to reduce toxicity when detected.

6. The method as claimed in claim 1, wherein the Fas-mediated apoptosis inducing agents comprise agonistic Fas antibodies and soluble FasL molecules.

7. The method as claimed in claim 6, wherein the soluble FasL molecule is selected among multimerized molecules of the soluble, extracellular domain of FasL.

8. The method as claimed in claim 7, wherein the soluble extracellular fraction of a Fas-L comprises amino acids Glu 139 to leu 281 of hFasL.

9. The method as claimed in claim 7, wherein the multimerization moiety comprises amino acids 17 to 110 of mACRP30 or amino acids 15 to 107 of hACRP30.

10. The method as claimed in claim 1, wherein the product preventing TNF receptors-mediated apoptosis of the liver cells is an anti-TNF/TNFR interaction product selected among anti-TNF antibodies and soluble TNF receptors.

11. The method as claimed in claim 10, wherein the anti-TNF antibody is Infliximab.

12. The method as claimed in claim 10, wherein the soluble receptor is Etanercept.

13. The method as claimed in claim 1, wherein the product preventing TNF-mediated apoptosis of the liver cells is a compounds interfering with TNF functions, preferably. thalidomide.

14. Pharmaceutical compositions comprising a Fas-mediated apoptosis inducing agent and a product preventing TNF receptors-mediated apoptosis of the liver cells.

15. Treatment kit comprising in separate pharmaceutical compositions a Fas-mediated apoptosis inducing agent and product preventing TNF receptors-mediated apoptosis of the liver cells.
